(19) United States
(12) Patent Application Publication
(10) Pub. No.: US 2005/0129573 A1
(43) Pub. Date: Jun. 16, 2005

(54) Carbon dioxide nanoelectronic sensor

(75) Inventors: Jean-Christophe P. Gabriel, Pinole, CA (US); George Gruner, Los Angeles, CA (US); Alexander Star, Albany, CA (US); Joseph R. Stetter, Hayward, CA (US)

Correspondence Address: Brian M. Berliner;O'MELVENY & MYERS LLP, 400 South Hope Street, Los Angeles, CA, 90071-2899, US

(73) Assignee: NANOMIX, INC.

(21) Appl. No.: 10/940,324

(22) Filed: Sep. 13, 2004

Related U.S. Application Data
(60) Provisional application No. 60/502,485, filed on Sep. 12, 2003.
(60) Provisional application No. 60/504,663, filed on Sep. 18, 2003.

(51) Int. Cl. G01N 33/00

(52) U.S. Cl. 422/58; 436/133

(57) ABSTRACT

An electronic system and method for detecting carbon dioxide is provided, using a nanostructure sensing device (CO₂ sensor). The CO₂ sensor is made up of a substrate and a nanostructure disposed over the substrate. The nanostructure may comprise a carbon nanotube, or a network of nanotubes. Two conductive elements are disposed over the substrate and electrically connected to the nanotube. A gate electrode may be positioned opposite the nanostructure. A functionalization material reactive with carbon dioxide is disposed on CO₂ sensor, and in particular, on the nanotube. The CO₂ sensor may be connected to an electrical circuit, which will respond to changes in CO₂ concentration in the ambient sensor environment.

### CROSS-REFERENCE TO RELATED APPLICATION

This application claims priority pursuant to 35 U.S.C. § 119(e) to U.S. provisional application Ser. No. 60/502,485, filed Sep. 12, 2003, and to provisional application Ser. No. 60/504,663, filed Sep. 18, 2003, which applications are specifically incorporated herein, in their entirety, by reference.

### BACKGROUND OF THE INVENTION

1. Field of the Invention

The present invention relates to nanostructure devices, such as nanotube sensors and transistors, and methods for fabricating the same.

2. Description of Related Art

Single-walled nanotube (“SWNT”) devices, including field-effect transistors (“FET's”) and resistors, can be fabricated using nanotubes grown on silicon or other substrates by chemical vapor deposition from iron-containing catalyst nanoparticles with methane/hydrogen gas mixture at 900° C. Other catalyst materials and gas mixtures can be used to grow nanotubes on substrates, and other electrode materials and nanostructure configurations and have been described previously by Gabriel et al. in U.S. patent application Ser. No. 10/099,664, filed Mar. 15, 2002, and in U.S. patent application Ser. No. 10/177,929, filed Jun. 2, 2002, both of which are specifically incorporated herein, in their entirety, by reference. Currently, technology for constructing practical nanostructure devices is in its infancy. While nanotube structures show promise for use as sensor devices and transistors, current technology is limited in many ways.

One potential application for SWNT sensors is CO₂ sensing. Currently, state-of-the-art sensing of CO₂ in indoor air quality applications makes use of non-nanotube, relatively large, power-hungry infrared sensors. Size, cost, and power constraints result in only limited use of these sensors.

If smaller, lower power, and lower cost CO₂ sensors could be made, they could be used much more widely. For example, in building air management, greater use of CO₂ sensors could result in better control of heating and ventilation systems, and thus energy cost savings. Other exemplary potential applications for simple, disposable CO₂ sensors may include medical applications such as capnography, wherein carbon dioxide levels in respiration are measured during intensive care and anesthesia. The high cost and limitations of current CO₂ sensors restrict the use of capnography to high value, controlled environments, such as surgical wards. Inexpensive, disposable CO₂ sensors would not only reduce capnography costs, but would facilitate mobile and temporary monitoring and broaden the reach of the technology.

Accordingly, there is a need for a smaller, lower power, and lower cost CO₂ sensor for a variety of applications.

### SUMMARY OF THE INVENTION

An electronic system and method for detecting carbon dioxide is provided, using a nanostructure sensing device (CO₂ sensor). The CO₂ sensor comprises a substrate and a nanostructure disposed over the substrate. In an embodiment of the invention, nanostructure comprises a carbon nanotube. Two conductive elements are disposed over the substrate and electrically connected to the nanotube. A functionalization material reactive with carbon dioxide is disposed on CO₂ sensor, and in particular, on the nanotube. The CO₂ sensor may be connected to an electrical circuit, which will respond to changes in CO₂ concentration in the ambient sensor environment.

Carbon nanotube field-effect transistor devices (NTFETs) may be fabricated which exhibit device characteristics that respond to chemical analytes through charge transfer between the NTFET and the analytes. Such devices are typically most sensitive to the presence of strong charge donors and acceptors, but are relatively less sensitive to week Lewis acids or bases such as H₂, CO₂ and CH₄. Specific sensitivity can be achieved by employing recognition layers that induce chemical reactions with a targeted analyte, thereby measurably changing the NTFET device characteristics.

Recognition layers that preserve the semi-conductive or conductive properties may be selected from noncovalent materials, for example, polymer coatings. Such organic recognition layers provide synthetic versatility and can be chemically modified for sensitivity to CO₂. Polymers have the additional advantage of being readily processable using procedures such as spin coating, dip coating, drop casting, and microspotting. Microspotting, in particular, may be useful for fabrication of multiple sensor in a sensor array that is configured to respond to a variety of different analytes. Yet another advantage is that polymer coatings often modify the characteristics of NTFET devices, which can be monitored during processing for control of coating processes.

A more complete understanding of the CO₂ sensor will be afforded to those skilled in the art, as well as a realization of additional advantages and objects thereof, by a consideration of the following detailed description of the preferred embodiment. Reference will be made to the appended sheets of drawings which will first be described briefly.

### BRIEF DESCRIPTION OF THE DRAWINGS

FIG. 1 is a schematic view of a nanostructure device with a recognition layer specific to CO₂ gas.

FIGS. 2A and 2B are charts showing results from testing of packaged nanostructure devices in flow cells at different CO₂ concentrations.

FIG. 3 is a diagram showing an exemplary mixture of poly(ethylene imine) (PEI) and starch polymers suitable for a CO₂-selective recognition layer in a nanotube field-effect transistor sensor device.

FIG. 4 is a chart showing the response to CO₂ gas for an optimized PEI/starch coated nanotube network field-effect transistor sensor device.

### DETAILED DESCRIPTION OF THE PREFERRED EMBODIMENT

Embodiments of this invention include a new sensing technology for carbon dioxide (CO₂) that uses nanoelectronic components. A tiny, low-cost nanosensor chip can offer: (i) performance that matches or exceeds that of infrared technology, (ii) plug-and-play simplicity with both digital and analog control systems, and (ii) the small size and low power consumption needed for wireless integration.

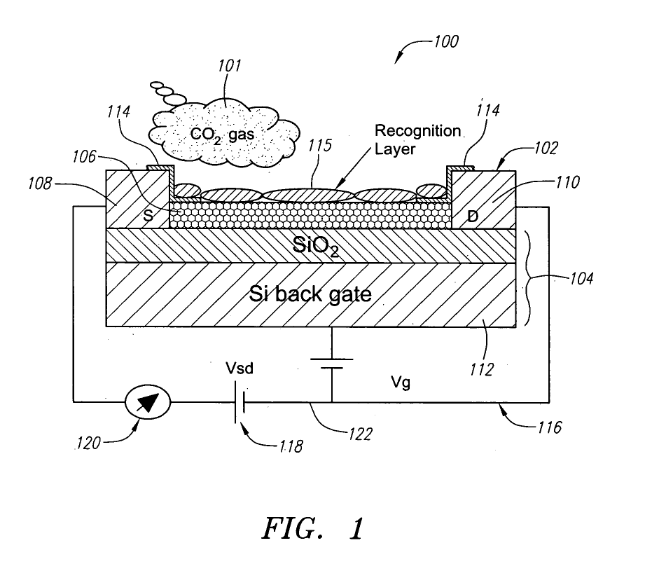

Field effect transistors made from semiconducting single-walled carbon nanotubes (NTFETs) have been used as the platform for sensitive chemical sensors. FIG. 1. shows an electronic system 100 for detecting carbon dioxide 101, comprising a nanostructure sensing device 102. Device 102 comprises a substrate 104, and a nanostructure 106 disposed over the substrate. The nanostructure may contact the substrate as shown, or in the alternative, may be spaced a distance away from the substrate, with or without a layer of intervening material. In an embodiment of the invention, nanostructure 106 may comprises a carbon nanotube. Any other suitable nanostructure, such as a nanowire, nanofiber, or nanorod, may also be used. In addition, or in the alternative, nanostructure 106 may comprise boron, boron nitride, and carbon boron nitride, silicon, germanium, gallium nitride, zinc oxide, indium phosphide, molybdenum disulphide, silver, or any other suitable material. In an alternative embodiment, nanostructure 106 comprises an interconnected network of smaller nanostructures. For example, nanostructure 106 may comprise a plurality of nanotubes forming a mesh.

Two conductive elements 108, 110 may be disposed over the substrate and electrically connected to nanostructure 106. Elements 108, 110 may comprise metal electrodes in direct contact with nanostructure 106. In the alternative, a conductive or semi-conducting material (not shown) may be interposed between elements 108, 110 and nanostructure 106. A functionalization material 115 reactive with carbon dioxide is disposed on nanostructure sensing device 102 and in particular, on nanostructure 106. Material 115 may be deposited in a continuous recognition layer, or in a discontinuous recognition layer. A suitable recognition layer may comprise more than one material and/or more than one layer of material.

Device 102 may further comprise a gate 112. Device 102 may further comprise a layer of inhibiting material 114 covering regions adjacent to the connections between the conductive elements 108, 110 and the first nanostructure 106. The inhibiting material may be impermeable to at least one chemical species, such as carbon dioxide. The inhibiting material may comprise a passivation material as known in the art, such as silicon dioxide. Further details concerning the use of inhibiting materials in a NTFET are described in prior application Ser. No. 10/280,265, filed Oct. 26, 2002, which is incorporated by reference herein.

In addition, system 100 may further comprise a second nanostructure sensing device (not shown) like device 102. It may be advantageous to provide the second device with a functionalization layer that incorporates a material different from that incorporated into layer 115.

System 100 may further include a nanostructure sensing device circuit 116. Circuit 116 may include one or more electrical supplies 118, a meter 120 in electrical communication with the electrical supply or supplies 118, and electrical connections 122 between the first nanostructure sensing device 102 and both the electrical supply and the meter. System 100 may further comprise a signal control and processing unit (not shown) as known in the art, in communication with the first nanostructure sensing device circuit.

The carbon nanotube acts not as the sensing element itself but as a sensitive transducer. There are various designs for the basic platform; these include devices with one or only a few nanotubes and devices with a network of nanotubes. A useful network of nanotubes may be provided, for example, by distributing a dispersion of nanotubes over a substrate so as to be approximately planar and randomly oriented. By distributing a large plurality of randomly oriented nanotubes in a dispersion over (or under) an electrode array, uniform electrical properties in the individual devices to be severed from the substrate can be assured with higher yields and faster processing than is possible using the prior art approach of controlled placement or growth of nanotubes. Further details of useful nanotube network device architectures, and methods for fabricating them, are described in the above-referenced application Ser. No. 10/177,929.

The nanotube transducers can be chemically functionalized to provide desired sensitivity and selectivity. They can even be made sensitive to a variety of inert gases. The functionalization approach relies on the ability of basic inorganic compounds and organic polymers as well as aromatic compounds with electron-donating functionalities to provide electrons to nanotubes, thus resulting in n-doping of NTFETs.

Sensitivity to CO₂ can be achieved through functionalization also. The functionalization layer has two main functions: 1) it selectively recognizes carbon dioxide molecules and 2) upon the binding of CO₂ it generates an amplified signal that is transferred to the carbon nanotube transducer. In the presence of water, carbon dioxide forms carbonic acid which dissociates and alters the pH of the functionalization layer, thus protonating the electron donating groups and making the NTFET more p-type.

Basic inorganic compounds (e.g., sodium carbonate), pH-sensitive polymers, such as polyaniline, poly(ethyleneimine), poly(o-phenylenediamine), poly(3-methylthiophene), and polypyrrole, as well as aromatic compounds (benzylamine, naphthalenemethylamine, antracene amine, pyrene amine, etc.) can be used to functionalize NTFETs for CO₂ sensing. The functionalization layer can be constructed using certain polymeric materials such as polyethylene glycol, poly(vinyl alcohol) and polysaccharides, including various starches as well as their components amylose and amylopectin. Other suitable materials for the functionalization layer may include, for example, metals, metal oxides, and metal hydroxides. In addition, a metallic functionalization layer may be combined with a polymeric functionalization layer.

Materials in the functionalization layer may be deposited on the NTFET using various different methods, depending on the material to be deposited. Metals can be evaporated on the sensor chip. In addition, or in the alternative, metals may be electroplated specifically on carbon nanotubes, for example, as described in more detail in the above-referenced provisional application Ser. No. 60/504,663.

Materials in the functionalization layer may be deposited on the NTFET using various different methods, depending on the material to be deposited. For example, inorganic materials, such as sodium carbonate, may be deposited by drop casting from 1 mM solution in light alcohols. The functionalized sensor may then be dried by blowing with nitrogen or other suitable drying agent. Polymeric materials may be deposited by dip coating. A typical procedure may involve soaking of the chip with the carbon nanotube device in 10% polymeric solution in water for 24 hours, rinsing with water several times, and blowing the chip dry with nitrogen. Polymers which are not soluble in aqueous solutions may be spin coated on the chip from their solutions in organic solvents. Values of polymer concentrations and the spin coater's rotation speeds may be optimized for each polymer.

CO₂ can be relatively unreactive and therefore more difficult to construct a sensor for. However, one useful reaction may be its combination with primary and secondary amines at ordinary temperatures and pressures to form carbamates. This may be exploited to form a NTFET sensor by using coating the nanotube portion of a sensor with a mixture of poly(ethylene imine) (PEI) and starch polymers. The sensing mechanism may involve adsorption of CO₂ in the polymer coating, followed by the establishment of an acid-base equilibrium involving water and PEI amino groups. The adsorption of CO₂ lowers the total pH of the polymer layer and alters the charge transfer to the semiconducting nanotube channel, resulting in the change of NTFET electronic characteristics.

While this sensing mechanism is based on a polymer coating of PEI or similar materials, for example polymers capable of adsorbing CO₂ from a gas mixture and having amino groups for facilitating the carbamate reaction, it may be greatly enhanced by the addition of a compatible hygroscopic material to the polymer layer. For example, a suitable reaction layer may be formed from a combination of PEI or similar polymer with a starch polymer. A suitable starch may include, for example, a mixture of linear component amylose and branched component amylopectin. It is believed that the presence of the starch attracts water, which in turn interacts with CO₂ and shifts the equilibrium due to the competing formation of carbonate and bicarbonate ions. A consequent increase in the local CO₂ concentration in the polymer recognition layer may result in larger protonation of PEI amino groups, and therefore provide the NTFET with a more sensitive response to CO₂.

Use of a recognition layer using PEI or similar polymer with a NTFET as described herein should result in an n-type sensor device. This effect may be attributed to electron-donating property of amino groups in the polymer. Carbamate formation reduces the overall electron donating effect of the polymer, leading to a device characteristic consistent with removal of electrons. Upon carbamate formation, geometric deformations may occur in the polymer layer leading to scattering sites on the nanotube and reduced conductance at positive gate voltages.

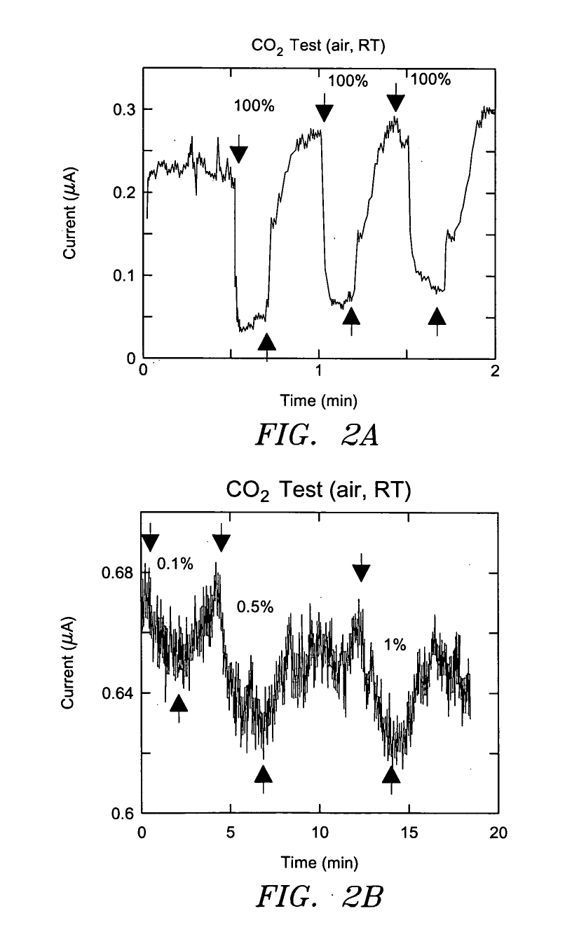

Measurements were made on packaged devices tested in a flow cell at controlled humidity and at different concentrations of CO₂ gas balanced with air. Exemplary responses of a PEI/starch functionalized NTFET device to carbon dioxide in this setup are shown in FIGS. 2A-B. Functionalized NTFET devices have showed reliable responses to CO₂ gas in air at ambient conditions as low as 1000 ppm. FIG. 2A shows the response of a functionalized NTFET sensor to a CO₂ concentration that cycled between 100% and 0%. FIG. 2B shows the response of a functionalized NTFET sensor to a CO₂ concentration that cycled between 0.1% and 0%, 0.5% and 0%, and 1% and 0%, in the sequence indicated on the figure.

Response and recovery times for a CO₂ sensor as described above may become progressively slower with each exposure, perhaps because of CO₂ saturation at the polymer/nanotube interface. It may be possible to reverse this slowing by sweeping the gate voltage between I-V_{G} measurements. Sweeping of the gate voltage may interfere with BCO₂⁻BH⁺ charges formed upon CO₂ binding in the polymer layer and thereby shift the equilibrium towards the original NTFET characteristic.

Other details concerning making and configuring NTFET's for use as chemical sensors may be as described in application Ser. No. 10/656,898, filed Sep. 5, 2003, which is incorporated by reference herein.

To improve sensor characteristics, a polymer recognition layer may be optimized for sensor performance by changing the ratio of polymers, deposition conditions and resulting polymer layer thickness. Modifications in the sensor platform may also be made to optimize the transducer electronic characteristics and response to CO₂ gas. For example, use of a nanotube network between the electrodes may provide more reproducible electronic characteristics before and after deposition of the recognition layer. Further details should be apparent from the examples below.

### EXAMPLES

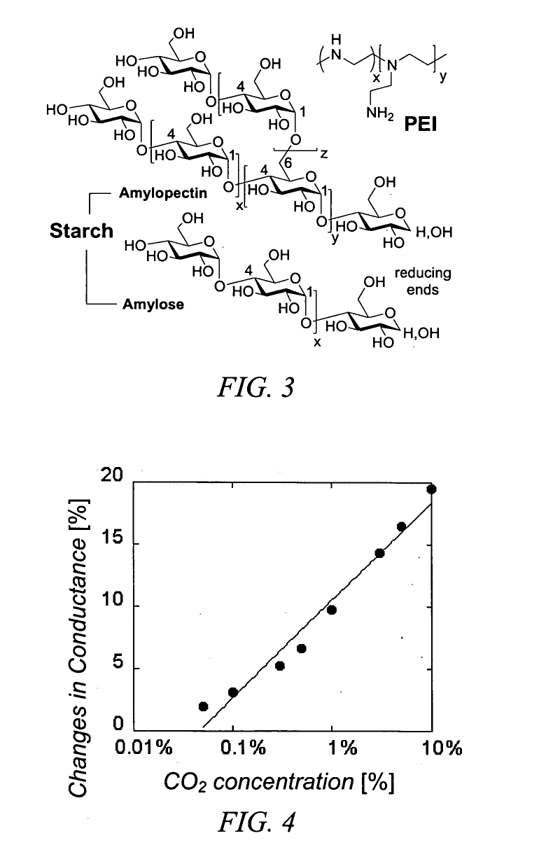

A mixture of poly(ethylene imine) (PEI) and starch polymers as diagrammed in FIG. 3 was used for the CO₂ selective recognition layer in nanotube field-effect transistor (NTFET) and nanotube network field-effect transistor (NTNFET) sensor devices. PEI, a highly branched polymer with 25% primary, 50% secondary, and 25% tertiary amino groups, can effectively adsorb CO₂ from the gas mixture. A combination of PEI and starch polymers in the CO₂ recognition layer is desired. Starch, a mixture of linear component amylose and branched component amylopectin, interacts strongly with nanotubes and affects CO₂ reaction with PEI amino groups.

In order to improve the required sensor characteristics, the polymer layer was optimized for sensor performance by changing the ratio of polymers, deposition conditions and resulting polymer layer thickness. Modifications in the sensor platform were also made to optimize the transducer electronic characteristics and its subsequent response to CO₂ gas. For example, the use of nanotube networks between the electrodes not only retains the field-effect transistor (FET) behavior, but also results in more reproducible electronic characteristics before and after recognition layer deposition. FIG. 4 shows the response to CO₂ gas for an optimized PEI/starch coated NTNFET sensor. The response to CO₂ gas is fast and reproducible at low concentrations, and has a wide dynamic range for CO₂ concentrations from 500 ppm to 10% in air.

NTFET and NTNFET devices were prepared according to published procedures, using standard photolithography techniques on 100 mm wafers. NTFET devices were fabricated using SWNTs grown by chemical vapor deposition (CVD) at 900° C. using dispersed iron nanoparticles as growth promoter and a methane/hydrogen gas mixture. Electrical leads were patterned on top of the nanotubes from titanium films 30 nm thick capped with a gold layer 120 nm thick. After conducting initial electrical measurements to establish the device characteristic, the substrates were submerged in a 10 wt % solution of poly(ethylene imine), PEI (average molecular weight ˜25,000, Aldrich chemicals) and starch (average molecular weight 10,000, Aldrich chemicals) in water overnight, followed by thorough rinsing with water. A thin layer (<10 nm) of polymer material coated the devices, as observed by atomic force microscopy.

For CO₂ sensing studies, chips with multiple NTFET devices were wire bonded and packaged in a 40-pin CERDIP package before functionalization with PEI/starch polymers. The polymer functionalized packaged devices were assembled in a flow cell in which air or CO₂ gas mixtures could be introduced to the devices. The low concentrations of CO₂ were achieved by mixing different proportions of air and 10% CO₂ in air with a CSSI 1010 precision gas diluter (Custom Sensor Solutions, Inc., Naperville, Ill.).

Having thus described a preferred embodiment of a carbon dioxide nanoelectronic sensor, it should be apparent to those skilled in the art that certain advantages of the within system have been achieved. It should also be appreciated that various modifications, adaptations, and alternative embodiments thereof may be made within the scope and spirit of the present invention. For example, a sensor making use of nanotubes has been illustrated, but it should be apparent that the inventive concepts described above would be equally applicable to other types of electro-responsive nanostructures. For example, similar structures may be constructed using nanowires or nanorods instead of NTFET's. In addition, the invention can be carried out by different equipment, materials and devices, and that various modifications, both as to the equipment and operating procedures, can be accomplished without departing from the scope of the invention. The invention is defined by the appended claims.

1. A nanostructure sensor for sensing carbon dioxide, comprising:
a substrate;
a first nanostructure over the substrate;
at least two conducting elements in electrical communication with the first nanostructure; and
at least one recognition material operatively associated with the first nanostructure, the at least one recognition material configured for interacting with carbon dioxide.

2. The nanostructure sensor of claim 1, wherein the first nanostructure is selected from the group consisting of nanotubes, nanowires, nanofibers, and nanorods.

3. The nanostructure sensor of claim 1, wherein the first nanostructure comprises at least one element selected from the group consisting of carbon, boron, boron nitride, and carbon boron nitride, silicon, germanium, gallium nitride, zinc oxide, indium phosphide, molybdenum disulphide, and silver.

4. The nanostructure sensor of claim 1, wherein the first nanostructure comprises a single-wall carbon nanotube.

5. The nanostructure sensor of claim 1, wherein the conducting elements comprise metal electrodes.

6. The nanostructure sensor of claim 1, wherein the conducting elements are in direct physical contact with the first nanostructure.

7. The nanostructure sensor of claim 1, wherein the at least one recognition material comprises a metallic carbonate.

8. The nanostructure sensor of claim 1, wherein the at least one recognition material is selected from the group consisting of lithium carbonate, sodium carbonate, potassium carbonate, cesium carbonate, barium carbonate, calcium carbonate, and silver carbonate.

9. The nanostructure sensor of claim 1, wherein the at least one recognition material comprises a pH-sensitive polymer.

10. The nanostructure sensor of claim 1, wherein the at least one recognition material is selected from the group consisting of polyaniline, poly(ethyleneimine), poly(o-phenylenediamine), poly(3-methylthiophene), and polypyrrole.

11. The nanostructure sensor of claim 1, wherein the at least one recognition material comprises an aromatic compound.

12. The nanostructure sensor of claim 1, wherein the at least one recognition material is selected from the group consisting benzylamine, naphthalenemethylamine, antracene amine, and pyrene amine.

13. The nanostructure sensor of claim 1, wherein the at least one recognition material comprises a polymeric material.

14. The nanostructure sensor of claim 1, wherein the at least one recognition material is selected from the group consisting of polyethylene glycol, poly(vinyl alcohol), polysaccharides, and starches.

15. The nanostructure sensor of claim 1, wherein the at least one recognition material comprises a substantially continuous layer over the nanostructure.

16. The nanostructure sensor of claim 1, wherein the at least one recognition material comprises a plurality of different materials.

17. The nanostructure sensor of claim 1, further comprising a gate electrode in proximity to the nanostructure.

18. The nanostructure sensor of claim 1, further comprising a layer of inhibiting material covering regions of the sensor adjacent to the connections between the conductive elements.

19. The nanostructure sensor of claim 1, wherein the nanostructure further comprises a two-dimensional nanostructure network disposed over the substrate between the two conduction elements.

20. The nanostructure sensor of claim 19, wherein the nanostructure network comprises a plurality of randomly-oriented carbon nanotubes.

21. The nanostructure sensor of claim 1, wherein at least one recognition material is selected from the group consisting of a metal, a metal oxide, and a metal hydroxide.

22. The nanostructure sensor of claim 1, wherein at least one recognition material comprises a layer of metal disposed adjacent the first nanostructure.

23. The nanostructure sensor of claim 22, wherein the recognition material includes a layer of polymeric material disposed adjacent the layer of metal.

24. The nanostructure sensor of claim 17, wherein at least one recognition material comprises a layer of metal disposed adjacent the gate electrode.

25. The nanostructure sensor of claim 24, wherein the recognition material includes a layer of polymeric material disposed adjacent the layer of metal.

26. The nanostructure sensor of claim 1, wherein the recognition material comprises poly(ethylene imine) in mixture with a starch.

27. The nanostructure sensor of claim 26, wherein the starch comprises at least one of amylose and amylopectin.
